(19) United States
(12) Patent Application Publication
(10) Pub. No.: US 2008/0221340 A1
(43) Pub. Date: Sep. 11, 2008

(54) Process for the Production of Nebivolol

(75) Inventors: Christian R. Noe, Vienna (AT); Muhamed Jasic, Vienna (AT); Hermann Kollmann, Linz (AT); Bodo Lachmann, Frankfurt (DE)

Correspondence Address: YOUNG & THOMPSON, 209 Madison Street, Suite 500, ALEXANDRIA, VA, 22314, US

(73) Assignee: Pharmacon Forschung und Bearatung GmbH, Wirn (AT)

(21) Appl. No.: 11/996,370

(22) PCT Filed: Jul. 17, 2006
(86) PCT No.: PCT/AT06/00303

§ 371 Date: Mar. 19, 2008

(30) Foreign Application Priority Data
Jul. 19, 2005 (AT) 1214/2005

(51) Int. Cl. C07D 311/02

(52) U.S. Cl. 549/407

(57) ABSTRACT

The subject of the invention is a process for the production of the racemic active ingredient nebivolol, in which diastereomeric cyanohydrins are produced, separated, and the separated diastereomers are coupled to one another after a transformation, preferably a partial or complete reduction of the cyano group or a Pinner saponification.

The invention relates to a process for the production of the racemic pharmaceutical substance nebivolol as a free base or as a pharmaceutically compatible salt, preferably as hydrochloride.

The pharmaceutical substance nebivolol of formula I,

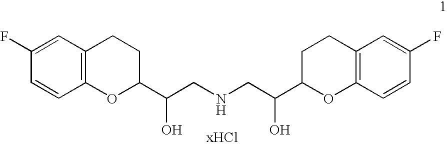

which is marketed as hydrochloride, belongs to the pharmaceutical substance group of the beta-receptor blockers. The molecule has 4 chirality centers and at the same time is structured centrosymmetrically. The approved pharmaceutical substance nebivolol is used as a racemate. Because of its symmetrical structure, instead of 8 diastereomers (from theoretically 16 enantiomers), only 6 diastereomers (R*R*R*R*, R*R*S*S*, R*R*R*S*, R*R*S*R*, R*S*R*S* and R*S*S*R*) with 10 isomers are formally to be considered in principle. Of the latter, the pure diastereomer with the designation R*R*R*S*, which is a 1:1 mixture of enantiomers with the absolute configurations RRRS and SSSR, is the actual approved active ingredient. A quite particularly special situation is produced from the fact that both enantiomers of the nebivolol with a different profile contribute synergistically to its pharmacological action. Therefore, as the highest possible standard, not the directed synthesis of a pure enantiomer but rather the synthesis of the racemic diastereomer with the desired relative configuration of the chirality centers can be considered.

In the literature, no immediately usable synthesis for the racemic nebivolol itself is found. To be sure, several syntheses for the components are described, but for separation into the individual stereoisomers, in each case chromatographic steps are used (e.g., EP 145067), whose description is kept unclear so that a usability that is technical in principle cannot be derived therefrom. Remarkably, a relatively large number of more or less advantageous syntheses for the pure enantiomer of nebivolol are found (e.g., S. Chandrasekhar, V. Reddy, Tetrahedron, 56 (2000), 6339-6344; C. W. Johannes, M. S. Visser, G. S. Weatherhead, A. H. Hoveyda, J. Am. Chem. Soc. 120 (1998), 8340-8347). A transfer of these syntheses for the production of the racemic active ingredient would require the separate production of enantiomers, however, which then must be mixed at 1:1. For the purpose of synthesis planning, the production of the racemic active ingredient is therefore more expensive than that of the pure enantiomer.

Nebivolol is—apart from the central element—formally built up of two molecule portions that are identical apart from their relative stereochemistry. The relative stereochemistry of the two chirality centers thereof is referred to as R*R* and R*S*. Notwithstanding the actual relative configuration, the diastereomeric intermediate products are differentiated below with A and B relative to their different configurations. Apart from a total unselective synthesis of nebivolol, in which all possible stereoisomers are formed and separated, strategies are offered in which the synthesis is carried out by linking two intermediate products with relative stereochemistry A and B of the chirality centers.

The subject of this invention is a process for the production of nebivolol, in which racemic diastereomeric cyanohydrins—diastereomers A and B below—of general formula 2

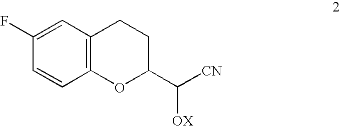

are used as intermediate products, in which X has the meaning of X═H, or X means a protective group that is typical of cyanohydrins, for example a benzylic (e.g., benzyl or 4-methoxybenzyl) or acetalic protective group (e.g., tetrahydropyranyl, or MEM), preferably a silyl protective group, and quite especially preferably a tert-butyldimethylsilyl protective group.

Other advantageous embodiments of the process according to the invention are disclosed according to subclaims.

The invention furthermore relates to (x-[(tert-butyldimethylsilyl)oxy]-6-fluoro-3,4-dihydro-2H-2-[1]benzopyran acetonitrile as a crystalline, racemic diastereomer A.

The invention also relates to α-[(tert-butyldimethylsilyl)oxy]-6-fluoro-3,4-dihydro-2H-2-[1]benzopyran acetonitrile as an oily, racemic diastereomer B.

The cyanohydrins can be produced from the aldehyde 3

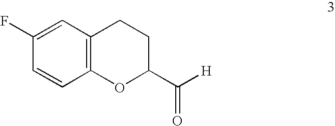

by a cyanohydrin reaction.

The aldehyde 3 can also be obtained by means of, i.a., ester 4 by reduction of the pyran ring, then direct or indirect (that is by means of alcohol) reduction of the ester group to form aldehyde.

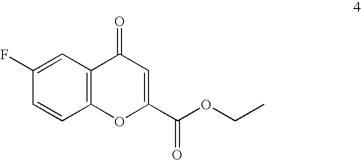

The cyanohydrin reaction can be performed stereoselectively or non-stereoselectively, whereby in the preferred variants, a non-stereoselective synthesis is carried out with O-derivatization. Diastereomers of the compound of formula 2, which carry silyl protective groups, can also be produced in one step with the aid of a corresponding silyl cyanide (tert-butyldimethylsilyl cyanide is preferred) from the aldehyde 3. The additional synthesis of the components before coupling is introduced with the separation of the diastereomeric cyanohydrins 2 with configurations A and B, which are both required for the additional synthesis. Especially preferred in terms of the invention are derivatized diastereomers A and B, which have clearly different crystallization properties, preferably those in which one diastereomer is present in crystalline form and the other is oily. In this way, a technologically quite especially simple and efficient separation of the diastereomers is made possible by digestion in an organic solvent. The tertbutyldimethylsilyl protective group is quite especially preferred in terms of the invention. One of the two diastereomers (A) is especially well crystallized, while the other diastereomer (B) is obtained as an oil, from which when using an apolar solvent, preferably a lower alkane, in particular hexane, small portions of A can also be easily separated while being cooled by crystallization. The crystalline diastereomer can be brought to a higher purity by recrystallization from an apolar solvent, preferably a lower alkane, in particular hexane.

For preparation of the linkage, the two molecule portions with configuration A or B are further reacted in various ways. The use of cyanohydrins as intermediate products proves especially advantageous for this purpose, since the cyanohydrin group can be further reacted in various ways to obtain optimum conditions for the linkage of the two molecule portions (A and B). Preferred reactions for further reaction are: the reduction to form O-protected aldehyde of general formula 5, in which X has the meaning that is indicated for formula 2, as well as the reduction to form the O-protected or O-unprotected amino alcohol of general formula 6, in which X has the meaning that is indicated for the formula 2, and the Pinner saponification to form the O-unprotected hydroxy esters of general formula 7, in which R means branched or unbranched lower alkyl or substituted or unsubstituted benzyl.

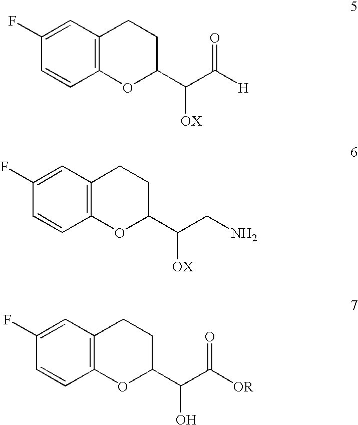

Both the crystalline cyanohydrin A of formula 2 and the oily cyanohydrin B of formula 2 can be reduced one time to form aldehyde 5 or to form hydroxyester 7 and the other time to form amine 6. In this case, differences in the yields are produced in the production and implementation of the subsequent reaction sequences.

The linkage and reaction to form nebivolol is preferably carried out by reaction of an aldehyde with an amine in a reductive amination, via Schiff base formation with subsequent reduction, preferably in a single-pot reaction with use of a complex hydride with limited reduction force, such as sodium cyanoborohydride or sodium triacetoxy borohydride, to form a compound of general formula 8,

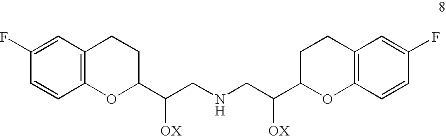

in which X has the meaning that is given for Formula 2,
or it is carried out by reaction of an ester of general formula 7 with an amine of general formula 6 to form the amide of general formula 9, in which X has the meaning that is indicated for the compound 2

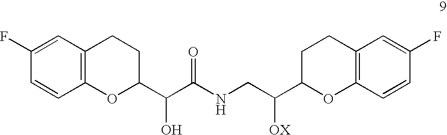

and subsequent reduction of the amide 9—via the amine of general formula 10, in which X has the meaning that is indicated for the compound 2.

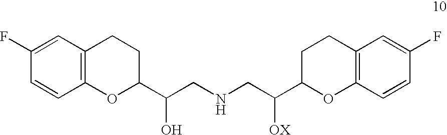

Nebivolol is obtained from the compounds of formulas 8 and 10, optionally after prior purification, by cleavage of protective groups, and it is purified optionally by recrystallization of the hydrochloride and/or the free base to form the nebivolol base or a pharmaceutically suitable salt thereof in pharmaceutical quality.

The optional use of O-protected or O-unprotected derivatives in the coupling, in particular the coupling of an O-protected aldehyde with an O-unprotected amine, opens up the possibility to separate especially efficiently by-product diastereomers that optionally are present in small amounts after coupling has taken place by purification of the mono-protected nebivolol of formula 10. This synthesis variant is shown in the following formula diagram for the case X=tert-butyldimethylsilyl.

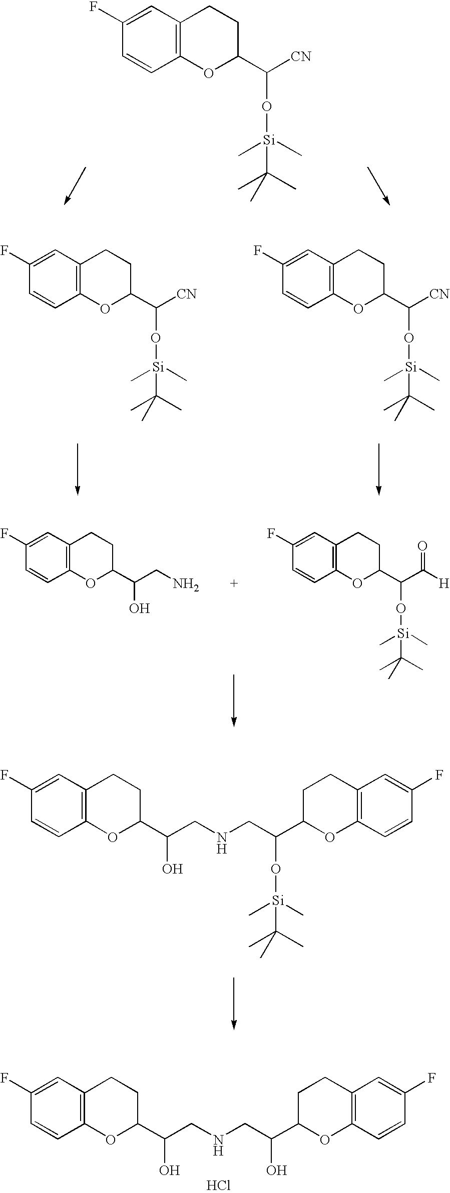

It is to be noted that in the coupling of a molecule with configuration A with one of configuration B, not a uniform diastereomer, but rather a mixture of two diastereomers is produced. In the coupling of a compound of the R*R* configuration with one of the R*S* configuration, namely the diastereomers R*R*R*S* (nebivolol, consisting of the enantiomers RRRS and SSSR) and R*R*S*R* (consisting of the enantiomers RRSR and SSRS) are produced. Actually, racemic nebivolol is a compound that crystallizes very readily both as a free base and as a hydrochloride. Especially advantageous is the fact that the diastereomer with the R*R*S*R* configuration that forms in the coupling in the 1:1 ratio can be removed especially easily in the recrystallization of the free base and the hydrochloride. Based on the significantly improved solubility properties of the undesired diastereomer, pure nebivolol can thus be obtained easily by recrystallization of the diastereomer mixture.

The invention is explained in more detail below based on possible embodiments for implementing the invention:

### EXAMPLE 1

### (R*,R*/R*,S*) 6-Fluoro-3,4-dihydro-α-hydroxy-2H-2-[1]benzopyran Acetonitrile—Racemic, Diastereomic Mixture

A solution of 33 g of (R*S*) 6-fluoro-3,4-dihydro-2H-[1]benzopyran-2-carbaldehyde in 150 ml of MTBE and 150 ml of 80% acetic acid is mixed with 23.8 g of potassium cyanide and stirred for 1 hour at room temperature under argon atmosphere. Then, the reaction mixture is added in drops into 600 ml of cooled, saturated aqueous sodium carbonate solution while being stirred slowly, and then solid sodium carbonate is added until the reaction solution reacts in a neutral manner. Then, it is extracted several times with MTBE, the organic phases are washed with water, dried with Na₂SO₄, mixed with one drop of phosphoric acid, and the solvent is removed in a vacuum.

Yield: 36.1 g (95%), yellowish oil. ¹H-NMR: (CDCl₃): δ (ppm)=6.82-6.74 (m, 3H), 4.20-4.14 (dd, 1H), 4.20-4.14 (m, 1H), 2.84-2.79 (m, 2H), 2.21-2.02 (m, 1H), 2.00-1.88 (m, 1H).

### EXAMPLE 2

### (R*,R*/R*,S*)-α-[(tert-Butyldimethylsilyl)oxy]-6-fluoro-3,4-dihydro-2-2H-[1]benzopyran Acetonitrile—Racemic, Diastereomer Mixture

Variant 1: 31.5 g of t-butyldimethylsilyl chloride is added at 0° C. to a solution of 25 g of imidazole in 150 ml of anhydrous dimethylformamide, and the mixture is stirred for 15 minutes at 0° C. Then, a solution of 36 g of cyanohydrin (Example 1) in 150 ml of anhydrous dimethylformamide is added in drops. After the addition is completed, it is stirred for 15 more minutes at 0° C., then brought to room temperature and stirred for 2 hours. Then, the reaction mixture is distributed between saturated NaHCO₃ solution and MTBE, the aqueous phase is extracted with MTBE, the combined organic phases are washed with water, dried on Na₂SO₄, and concentrated by evaporation in a vacuum. The DMF is scrubbed several times with toluene, and the product is dried under high vacuum.

Yield: 53.1 g (95%), yellow oil; ¹H-NMR: (CDCl₃): δ (ppm)=6.84-6.78 (m, 3H), 4.75/4.62 (dd, 1H), 4.25-4.07 (m, 1H), 2.89-2.85 (m, 2H), 2.37-2.17 (m, 1H), 2.03-1.85 (m, 1H), 0.97 (d, 9H), 0.23 (t, 6H).

Variant 2: With the Aid of tert-Butyldimethylsilyl Cyanide

1.77 g of zinc iodide is added to a solution of (R*S*) 6-fluoro-3,4-dihydro-2H-[l]benzopyran-2-carbaldehyde, 2 g, in 20 ml of anhydrous DCM. At −10° C., 1.88 g of tert-butyldimethylsilyl cyanide is added, and the mixture is stirred for 2 hours at this temperature. Then, the reaction mixture is distributed between 5% NaHCO₃ solution and MTBE, the aqueous phase is extracted twice with MTBE, the combined organic phases are washed with water, dried on Na₂SO₄, and concentrated by evaporation in a vacuum. The product is dried under high vacuum.

Yield: 3.1 g (87%), yellow oil.

¹H-NMR: (CDCl₃): δ (ppm)=6.83-6.79 (m, 3H), 4.73/4.61 (dd, 1H), 4.23-4.07 (m, 1H), 2.90-2.85 (m, 2H), 2.37-2.15 (m, 1H), 2.01-1.83 (m, 1H), 0.96 (d, 9H), 0.21 (t, 6H).

### EXAMPLE 3

### α-[(tert-Butyldimethylsilyl)oxy]-6-fluoro-3,4-dihydro-2H-2-[1]benzopyran Acetonitrile—Crystalline, Racemic Diastereomer A

16 g of the racemic diastereomer mixture from Example 2 is dissolved in 20× the amount of petroleum ether, brought to −45° C. and inoculated with an inoculation crystal of the crystalline diastereomer components. After 4 hours, the mother liquor is decanted off, the crystals are digested with a little deep-frozen petroleum ether, the latter is decanted off, and the combined mother liquors are concentrated by evaporation to one third of the volume of the original solution, inoculated again, and the described procedure is repeated, by which a second yield of product is obtained. A crystalline fraction of the R*,S*-diastereomer (residual content of R*,R*-diastereomer 8%), which is dissolved again in petroleum ether while being heated for further purification and is crystallized out at lower temperature, is obtained.

Yield: Crystal fraction: 6.5 g=40%, white crystals; flash point=66° C.

¹H-NMR: (CDCl₃): δ (ppm) 6.83-6.73 (m, 3H), 4.71 (d, 1H), 4.09-4.05 (m, 1H), 2.91-2.79 (m, 2H), 2.30-2.25 (m, 1H), 1.97-1.88 (m, 1H), 0.94 (s, 9H), 0.23 (s, 3H), 0.19 (s, 3H).

### EXAMPLE 4

### α-[(tert-Butyldimethylsilyl)oxy]-6-fluoro-3,4-dihydro-2H-2-[1]benzopyran Acetonitrile—Oily, Racemic Diastereomer B

After the concentration by evaporation of the mother liquor that is removed twice from the crystallization of diastereomer A, the diastereomer B (residual content of diastereomer=10%) is obtained; yield: 9.0 g=55%, yellow oil; ¹H-NMR: (CDCl₃): δ (ppm), 6.83-6.72 (m, 3H), 4.58 (d, 1H), 4.18-4.14 (m, 1H), 2.90-2.77 (m, 2H), 2.22-2.17 (m, 1H), 1.93-1.85 (m, 1H), 0.92 (d, 9H), 0.24 (s, 3H), 0.16 (s, 3H).

### EXAMPLE 5

### α-[(tert-Butyldimethylsilyl)oxy]-6-fluoro-3,4-dihydro-2H-2-[1]benzopyran Acetaldehyde—Racemic Diastereomer B

A solution of 8 g of the oily, racemic diastereomeric cyanohydrin B from Example 4 in 80 ml of toluene is brought in at 5° C., mixed with 18.25 ml of a 1.5 M solution of diisobutyl aluminum hydride in toluene. The reaction mixture is stirred for 1 hour at room temperature. Then, the reaction mixture is added to 600 ml of 1N hydrochloric acid and diluted with 100 ml of MTBE. The phases are separated, and the aqueous phase is extracted three more times with 200 ml each of MTBE. The combined organic phases are washed with saturated sodium chloride solution, dried on Na₂SO₄, and the solvent is removed in a vacuum.

Yield: 7.1 g (88%), yellow oil.

¹H-NMR: (CDCl₃): δ (ppm)=9.79 (s, 1H), 6.84-6.77 (m, 3H), 4.32-4.25 (m, 1H), 3.73-3.65 (m, 1H), 2.84-2.79 (m, 2H), 2.00-1.91 (m, 2H), 0.98 (d, 9H), 0.25 (t, 6H).

### EXAMPLE 6A

### 6-Fluoro-3,4-dihydro-α-hydroxy-2H-2-[1]benzopyran Ethanamine-Racemic Diastereomer A

20.7 ml of a 1.5 M DIBAL solution in toluene is added in drops at −20° C. to a solution of 5 g of the crystalline, racemic diastereomer A of α-[(tert-butyldimethylsilyl)oxy]-6-fluoro-3,4-dihydro-2H-2-[1]benzopyran acetonitrile in 50 ml of toluene. It is heated to room temperature and stirred for 15 minutes. Then, the reaction mixture is cooled to 0° C. and mixed with 1.18 g of lithium aluminum hydride. Then, the reaction mixture is stirred for 14 hours at room temperature. Then, another 20.7 ml of a 1.5M DIBAL solution in toluene is added, stirred for 1 hour at room temperature and for 1 hour at 35° C. The reaction mixture is added to a cooled solution of 400 ml of ethyl acetate and 20 ml of MeOH. To this end, 300 ml of potassium tartrate solution and 30 ml of 2N NaOH are added and stirred for 1 hour. This reaction mixture is filtered on Celite, and the residue and the Celite are rewashed three times with 150 ml of ethyl acetate each. The phases are separated, the organic phases are washed with water, dried, and the solvent is removed in a vacuum. 3.49 g of crystalline crude product is obtained. The latter is recrystallized from MTBE.

Yield: 2.15 g (65.5%), white solid; Flash point=124° C.

¹H-NMR: (CDCl₃): δ (ppm)=6.81-6.77 (m, 3H), 4.02-3.95 (m, 1H), 3.71-3.65 (m, 1H), 3.00-2.81 (m, 4H), 2.04-1.85 (m, 5H).

### EXAMPLE 6B

### 6-Fluoro-3,4-dihydro-α-hydroxy-2H-2-[1]benzopyran Ethanamine Racemic Diastereomer B

16.6 ml of a 1.5M DIBAL solution in toluene is added in drops at −40° C. to a solution of 4 g of the racemic diastereomer B of α-[(tert-butyldimethylsilyl)oxy]-6-fluoro-3,4-dihydro-2H-2-[1]benzopyran acetonitrile in 40 ml of toluene. It is heated to room temperature and stirred for 15 minutes. Then, the reaction mixture is cooled to 0° C. and mixed with 1.18 g of lithium aluminum hydride. Then, the reaction mixture is stirred for 14 hours at room temperature. Then, another 16.6 ml of a 1.5M DIBAL solution in toluene is added, stirred for 1 hour at room temperature and for 1 hour at 35° C. The reaction mixture is added to a cooled solution of 300 ml of ethyl acetate and 20 ml of MeOH. 250 ml of potassium tartrate solution and 25 ml of 2N NaOH are added to this and stirred for 1 hour. This reaction mixture is filtered on Celite, and the residue and the Celite are rewashed three times with 120 ml of ethyl acetate each. The phases are separated, the organic phases are washed with water, dried, and the solvent is removed in a vacuum. 2.4 g of crystalline crude product is obtained. The latter is recrystallized from MTBE.

Yield: 1.7 g (64.6%), white solid: ¹H-NMR: (CDCl₃): δ (ppm)=6.78-6.65 (m, 3H), 3.87-3.84 (m, 1H), 3.67-3.63 (m, 1H), 3.00-2.71 (m, 4H), 2.51 (bs, 1H), 2.14-2.10 (m, 1H), 1.84-1.76 (m, 1H).

### EXAMPLE 7

### N-{2-[(tert-Butyldimethylsilyl)oxy]-2-(6-fluoro-3,4-dihydro-2H-2-[1]benzopyranyl)ethyl]-6-fluoro-3,4-dihydro-α-hydroxy-2H-2[1]benzopyran Ethanamine—Racemic A/B Diastereomer

0.81 g of sodium cyanoborohydride is added to a solution of 2.28 g of the racemic diastereomer A of 6-fluoro-3,4-dihydro-α-hydroxy-2H-2-[1]benzopyran ethanamine and 7 g of the racemic diastereomer B of α-[(tert-butyldimethylsilyl)oxy]-6-fluoro-3,4-dihydro-2H-2-[1]benzopyran acetaldehyde in 250 ml of THF and 50 ml of methanol. 1 ml of glacial acetic acid is added, and it is stirred for 14 hours at room temperature. Then, another 0.81 g of sodium cyanoborohydride is added. It is stirred for another 18 hours at room temperature. The reaction mixture is mixed with 400 ml of 5% NaHCO₃ solution and extracted several times with ethyl acetate after 5 minutes of stirring. The organic phases are dried, and the solvent is removed in a vacuum. The thus obtained crude product (10.1 g) is purified by flash chromatography (mobile solvent DCM/MeOH 50+1). 4.2 g of yellow oil is obtained.

¹H-NMR: (CDCl₃): δ (ppm)=6.81-6.75 (m, 6H), 4.09-3.95 (m, 4H), 2.92-2.65 (m, 8H), 2.10-1.98 (m, 2H), 1.87-1.72 (m, 2H), 0.94 (s, 9H), 0.18 (s, 3H), 0.13 (s, 3H).

### EXAMPLE 8

### (R*,R*,R*,S*)-α,α′-[Iminobis(methylene)bis[6-fluoro-3,4-dihydro-2H-1-benzopyran-2-methanol]] Hydrochloride—Nebivolol

A solution of 4.2 g of the racemic A/B diastereomer N-{2-[(tert-butyldimethylsilyl)oxy]-2-(6-fluoro-3,4-dihydro-2H-2-[1]benzopyranyl)ethyl}-6-fluoro-3,4-dihydro-α-hydroxy-2H-2-[1]benzopyran ethanamine in 75 ml of THF and 75 ml of MeOH is mixed with 25 ml of 6N hydrochloric acid and stirred for 1 hour at 70° C. Then, it is concentrated by evaporation to one-half its original volume, diluted with 350 ml of water and extracted with petroleum ether. The aqueous phase is made basic with 2N NaOH and extracted several times with ethyl acetate. The combined organic phases are washed with water, dried, and the solvent is removed in a vacuum.

Yield: 3.2 g (97%), yellow oil.

The oil is taken up in 30 ml of methanol, mixed with 6 ml of 2N HCl in diethyl ether and allowed to stand at −20° C. for crystallization. Traces of diastereomeric contaminants can be separated from methanol by recrystallization.

Yield: 1.15 g (32.1%); ¹H-NMR: (MeOD): δ (ppm)=6.84-6.75 (m, 6H), 4.14-4.11 (m, 1H), 4.04-4.01 (m, 2H), 3.95-3.91 (m, 1H), 3.53 (dd, 1H), 3.44-3.34 (m, 2H), 3.26 (dd, 1H), 2.96-2.78 (m, 4H), 2.29-2.22 (m, 1H), 2.05-1.89 (m, 2H), 1.84-1.74 (m, 1H).

In summary, it can be stated that the racemic diastereomers of nebivolol with the desired relative configuration of the chirality centers are obtained with high yields and satisfactory degrees of purity by the process according to the invention. This can be done according to the invention insofar as the corresponding diastereomeric cyanohydrins are produced, separated, and the separated diastereomers are coupled to one another after a transformation, preferably a partial or complete reduction of the cyano group or a Pinner saponification.

1. Process for the production of the racemic pharmaceutical substance nebivolol as a free base or as a pharmaceutically compatible salt, preferably as hydrochloride according to formula 1 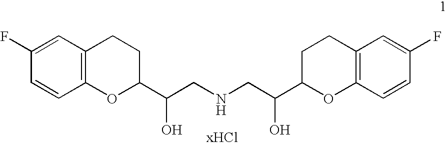
characterized in that the synthesis is accomplished via racemic, diastereomeric cyanohydrins—diastereomers A and B below—of general formula 2 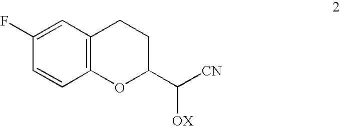
in which X is equal to hydrogen or X means a protective group that is typical of cyanohydrins.

2. Process according to claim 1, wherein the protective group X that is typical of cyanohydrins is a benzylic protective group, for example, a benzyl or 4-methoxybenzyl protective group, or an acetalic protective group, for example a tetrahydropyranyl or MEM protective group.

3. Process according to claim 1, wherein the protective group X that is typical of cyanohydrins is a silyl protective group, preferably a tert-butyldimethylsilyl protective group.

4. Process according to claim 1, wherein the diastereomers A and B are separated from one another by crystallization.

5. Process according to claim 4, wherein a crystalline diastereomer A is separated from a non-crystalline diastereomer by digestion.

6. Process according to claim 1, wherein for preparing the linkage for the production of nebivolol, the two diastereomers of general formula 2 are further reacted in various ways with the configurations A and B, whereby as reactions, the reduction to form the aldehyde of general formula 5 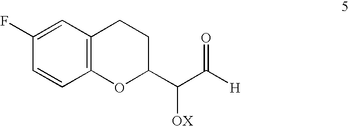
in which X has the meaning that is indicated for formula 2, as well as the reduction to form the amino alcohol of general formula 6, 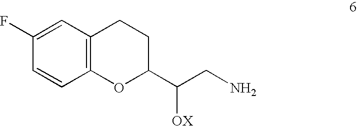
in which X has the meaning that is indicated for formula 2, and the Pinner saponification to form hydroxyl esters of general formula 7, 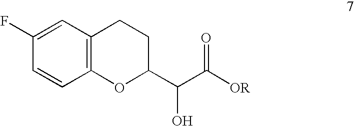
in which R means branched or unbranched lower alkyl, or substituted or unsubstituted benzyl, are preferred.

7. Process according to claim 6, wherein the reduction to form the aldehyde of general formula 5 is carried out with a complex hydride, for example with diisobutylaluminum hydride.

8. Process according to claim 6, wherein the reduction to form amine of formula 6 is carried out with a complex hydride, for example with lithium aluminum hydride, preferably in combination with diisobutylaluminum hydride.

9. Process according to claim 6, wherein the Pinner saponification to form hydroxy esters of formula 7 is carried out while being cooled at temperatures of below 10 degrees C. in dry ether, preferably diethyl ether, THF or MTBE, in the presence of an excess of alcohol ROH, and as an acid, hydrochloric acid is introduced as a gas or is added as a solution in ether.

10. Process according to claim 1, wherein in the synthesis sequence, the diastereomer B of general formula 2 is reduced to form the aldehyde of formula 5, in which X has the meaning that is given for formula 2, and the diastereomer A of general formula 2 is reduced to form the amino alcohol of formula 6, in which X=the meaning that is given in formula 2, and the two components are reacted by a reductive amination with one another to form the nebivolol of general formula 8 that is protected with protective groups X 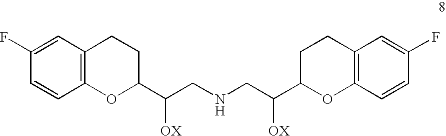
after which, optionally after prior purification, nebivolol is obtained by cleavage of the protective groups X and is purified to pharmaceutical quality optionally by recrystallization of the hydrochloride or another readily crystallizing salt and/or the free bases.

11. Process according to claim 1, wherein in the synthesis sequence, the diastereomer B of general formula 2 is reduced to form the oxygen-protected aldehyde of formula 5, in which X has the meaning that is given for formula 2, with the exception of hydrogen, and the diastereomer A of general formula 2 is reduced to form the amino alcohol of formula 6, in which X means the same as hydrogen, and the two components are reacted with one another by a reduction amination to form a nebivolol of general formula 10 that is protected with a protective group X.

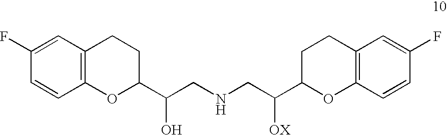

12. Process according to claim 10, wherein in the further reaction of the diastereomeric cyanohydrins A and B according to formula 2 to form nebivolol, the diastereomer A of general formula 2 is reduced to form the aldehyde of formula 5, and the diastereomer B of general formula 2 is reduced to form the amino alcohol of formula 6.

13. Process according to claim 1, wherein the further reaction of the diastereomeric cyanohydrins A and B of formula 2 is carried out to form nebivolol by reaction of an ester of general formula 7 with an amine of general formula 6 to form the amide of general formula 9, 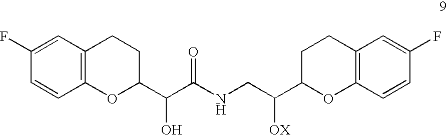
in which X has the meaning that is indicated for the compound 2, followed by a reduction to form the amine of general formula 10, 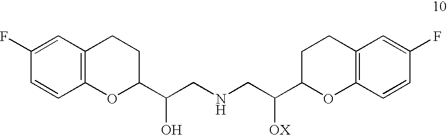
in which X has the meaning that is indicated for the compound 2, from which, optionally after prior purification, nebivolol is obtained by the cleavage of protective groups and is purified to pharmaceutical quality optionally by recrystallization of the hydrochloride or another readily crystallizing salt and/or free base.

14. Process according to claim 1, wherein for further reaction of the diastereomeric cyanohydrins A and B of formula 2 to form nebivolol, the diastereomer B of general formula 2 is reduced by Pinner saponification to form hydroxy esters of formula 7, and the diastereomer A of general formula 2 is reduced to form oxygen-protected amino alcohol of general formula 6.

15. Process according to claim 1, wherein for further reaction of the diastereomeric cyanohydrins A and B of formula 2 to form nebivolol, the diastereomer A of general formula 2 is reduced by Pinner saponification to form hydroxy esters of formula 7, and the diastereomer B of general formula 2 is reduced to form oxygen-protected amino alcohol of general formula 6.

16. Process for the production of cyanohydrins with general formula 2, wherein an aldehyde of formula 3 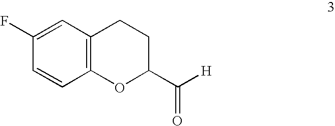
is reacted to form the corresponding cyanohydrin, and the protective group X, preferably a tert-butyldimethylsilyl protective group, is introduced.

17. α-[(tert-Butyldimethylsilyl)oxy]-6-fluoro-3,4-dihydro-2H-2-[1]benzopyran acetonitrile as a crystalline, racemic diastereomer A.

18. α-[(tert-Butyldimethylsilyl)oxy]-6-fluoro-3,4-dihydro-2H-2-[1]benzopyran acetonitrile as an oily, racemic diastereomer B.

19. Process according to claim 11, wherein in the further reaction of the diastereomeric cyanohydrins A and B according to formula 2 to form nebivolol, the diastereomer A of general formula 2 is reduced to form the aldehyde of formula 5, and the diastereomer B of general formula 2 is reduced to form the amino alcohol of formula 6.
